(19) United States
(12) Patent Application Publication
(10) Pub. No.: US 2010/0240566 A1
(43) Pub. Date: Sep. 23, 2010

(54) Washing or Cleaning Agents with Amidine Compounds and/or Amidinium Bicarbonates

(76) Inventors: Georg Meine, Mettmann (DE); Thomas Feldbrugge, Dortmund (DE); Paula Barreleiro, Dusseldorf (DE); Konstantin Benda, Dusseldorf (DE); Emilie Barriau, Dusseldorf (DE)

Correspondence Address: Henkel Corporation, 10 Finderne Avenue, Suite B, Bridgewater, NJ, 08807, US

(21) Appl. No.: 12/794,005

(22) Filed: Jun. 4, 2010

Related U.S. Application Data
(63) Continuation of application No. PCT/EP2008/063314, filed on Oct. 6, 2008.

(30) Foreign Application Priority Data
Dec. 5, 2007 (DE) 10 2007 058 846.3

(51) Int. Cl. C11D 3/26; C07C 257/10; C11D 17/00

(52) U.S. Cl. 510/276; 564/225; 510/499; 510/445

(57) ABSTRACT

Washing and cleaning agents containing a specific amidine compound and/or specific amidinium bicarbonate. These washing or cleaning agents enable an improved removal of contaminants due to pigment contamination and fat and oil soiling. Use of special amidine compounds and amidinium bicarbonate in a washing solvent is also described, which enables carbon dioxide from ambient air to bind in the washing solvent.

### CROSS REFERENCE TO RELATED APPLICATIONS

The present application is a continuation of International Application No. PCT/EP2008/063314 filed 6 Oct. 2008, which claims priority to German Patent Application No. 10 2007 058 846.3 filed 5 Dec. 2007.

The present invention relates to washing or cleaning agents which contain a specific amidine compound and/or specific amidinium bicarbonate. It additionally relates to a textile washing method using such washing or cleaning agents. It additionally relates to the use of such washing or cleaning agents in relation to manual or automatic laundering of textiles, in particular in relation to improving dirt detachment in the case of pigment dirt and in the case of grease and oil soiling. It furthermore relates to use of the specific amidine compound and/or of the specific amidinium bicarbonate in a washing liquor, in order to absorb carbon dioxide from the ambient air into the washing liquor and bind it therein.

Since at the latest 1907, when the first automatic washing agent was produced in the form of Persil®, washing or cleaning agents have been among those consumer goods which have a particular impact on making people's lives easier, better and more pleasant. Despite all the progress achieved thus far, the aim of manufacturers of washing or cleaning agents is to make the quality of their products ever better.

Given this general background, a very specific challenge is to provide washing or cleaning agents with improved cleaning performance with regard to engine oil-based soiling. In particular employees of auto shops, for example, find themselves having to deal with significant soiling of this type. Engine oil is the collective term for the mineral oils, and indeed synthetic oils with or without mineral oil additives, which are suitable for use as engine lubricants.

The object of the present invention was to overcome the above-stated specific challenge. It has wholly surprisingly now been found that it is possible to achieve this object precisely with washing or cleaning agents which contain specific amidine compounds and/or amidinium bicarbonates.

The present invention therefore provides a washing or cleaning agent containing an amidine compound according to Formula (1)—

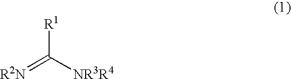

and/or an amidinium bicarbonate according to Formula (2)—

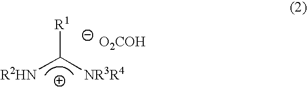

wherein R¹, R², R³, R⁴ are mutually and independently C₁-C₂₅ hydrocarbon residues or H. The saturated or unsaturated, optionally substituted C₁-C₂₅ hydrocarbon residues may be straight-chain, cyclic or branched. (This basic statement about hydrocarbon residues also applies below in relation to amidine compounds according to Formula (1) and amidinium bicarbonates according to Formula (2) but is not mentioned again so as to avoid repetition).

Preferably, at least one of the residues R¹, R², R³ or R⁴, in particular the residue R², is a C₆C₁₈ hydrocarbon residue, preferably C₈-C₁₈ hydrocarbon residue, the other residues R¹, R², R³ or R⁴, which are not C₆-C₁₈ hydrocarbon residues, being mutually independently preferably C₁-C₄ hydrocarbon residues or H. If the residue R² denotes a C₁-C₁₈ hydrocarbon residue in the amidine compound (1) and/or in the amidinium bicarbonate (2), in particular R² is a saturated, straight-chain C₈-C₁₈ hydrocarbon residue, and the residues R¹, R³ and R⁴ are mutually independently hydrogen residues, methyl or ethyl residues, preferably methyl residues, a preferred embodiment of the invention is provided.

It has wholly unexpectedly been found that, when washing or cleaning agents according to the invention are used, a very marked, unforeseen improvement in performance occurs with regard to the removal in particular of engine oil-based soiling, in particular in the case of manual or automatic laundering of textiles. It is particularly advantageous that it is not only the removability of engine oil-based soiling which is improved but also moreover the removability of other soiling, such as in particular pigment-containing and fat- or oil-containing soiling, is unexpectedly improved. This applies in particular to the removability of soiling attributable in particular to garden soil, makeup or sebaceous matter.

Preference is very particularly given to those washing or cleaning agents according to the invention which contain at least one amidine compound according to Formula (1) as previously described.

In a preferred embodiment of the invention, a washing or cleaning agent according to the invention contains 0.01 wt. % to 25 wt. %, preferably 0.1 to 20 wt. %, advantageously 0.5 to 10 wt. %, in particular 1 wt. % to 5 wt. % of an amidine compound of the formula (1) and/or of an amidinium bicarbonate according to the formula (2), wt. % relating to the total agent.

It is particularly preferable for the washing or cleaning agent according to the invention additionally to contain anionic, nonionic and/or cationic surfactants, in particular however a mixture of anionic and nonionic surfactants, in which the total agent preferably contains 0.1 wt. % to 50 wt. %, in particular 10 wt. % to 40 wt. % of surfactant, relative to the total agent. This corresponds to a preferred embodiment of the invention and allows optimum cleaning performance, in particular with regard to engine oil-related soiling. Surfactants which may preferably be used are described further below in greater detail.

It is particularly favorable for the washing or cleaning agent according to the invention to contain alkylbenzene sulfonate, preferably linear alkylbenzene sulfonate (LAS), preferably in quantities of from 0.1-25 wt. %, more advantageously 1-20 wt. %, in particular in quantities of from 5-15 wt. %, relative to the total agent. This corresponds to a preferred embodiment of the invention and allows very particularly advantageous cleaning performance, in particular with regard to the removability of soiling attributable to engine oil.

Particularly suitable anionic surfactants are furthermore alkyl sulfates, in particular fatty alcohol sulfates (FAS), for example C₁₂-C₁₈ fatty alcohol sulfate. C₈-C₁₆ alkyl sulfates may preferably be used, with C₁₋₃ alkyl sulfate and C₁₃₋₁₅ alkyl sulfate and C₁₃₋₁₇ alkyl sulfate being particularly preferred, advantageously branched, in particular alkyl-branched C₁₃₋₁₇ alkyl sulfate. Particularly suitable fatty alcohol sulfates are derived from lauryl and myristyl alcohol (i.e., are fatty alcohol sulfates with 12 or 14 carbon atoms). The long-chain FAS types (C₁₆ to C₁₈) are very well suited to washing at higher temperatures. Alkyl sulfates are particularly preferred which have a lower Krafft point, preferably a Krafft point lower than 45, 40, 30 or 20° C.

Krafft point is the name given to that temperature at which the solubility of surfactants rises sharply as a result of the formation of micelles. The Krafft point is a triple point at which the solids content or hydrated crystals of the surfactant are at equilibrium with the dissolved (hydrated) monomers and micelles thereof. The Krafft point is determined using a turbidity measurement according to DIN EN 13955: 2003-03.

If the washing or cleaning agent according to the invention contains alkyl sulfate, in particular C₁₂-C₁₈ fatty alcohol sulfate, advantageously in quantities of from 0.1-25 wt. %, more advantageously 1-20 wt. %, in particular in quantities of from 5-15 wt. %, relative to the total agent, a preferred embodiment of the invention is provided.

The specific combination of FAS and/or LAS with the amidine compound to be used according to the invention of the formula (1) and/or the amidinium bicarbonate of the formula (2) in a washing or cleaning agent according to the invention leads wholly unexpectedly, both in the case of natural and synthetic greases, to a very good cleaning result, the FAS-containing washing or cleaning agents displaying particularly good results with regard to the removal of sebaceous dirt and natural greases.

Other suitable anionic surfactants which may likewise be used are for example alkane sulfonates (for example secondary C₁₃-C₁₈ alkane sulfonate), methyl ester sulfonates (for example α-C₁₂-C₁₈ methyl ester sulfonate) and α-olefin sulfonates (for example α-C₁₄-C₁₈ olefin sulfonate) and alkyl ether sulfates (for example C₁₂-C₁₄ fatty alcohol-2EO-ether sulfate) and/or soaps. Further suitable anionic surfactants are described further below in greater detail. FAS and/or LAS are particularly suitable, however.

If the washing or cleaning agent according to the invention comprises nonionic surfactant, in particular alkoxylated fatty alcohol, in particular in quantities of from 0.01-20 wt. % relative to the total agent, a preferred embodiment of the invention is in turn provided in which the combined use of alkylbenzene sulfonate with nonionic surfactant is very particularly preferred.

Further suitable nonionic surfactants are alkyl phenol polyglycol ethers (APEO), (ethoxylated) sorbitan fatty acid esters (sorbitans), alkyl polyglucosides (APG), fatty acid glucamides, fatty acid ethoxylates, amine oxides, ethylene oxide/propylene oxide block polymers, polyglycerol fatty acid esters and/or fatty acid alkanolamides. Further suitable nonionic surfactants are described further below in greater detail. Sugar-based nonionic surfactants, such as in particular APG, are particularly preferred.

According to a further preferred embodiment of the invention, the washing or cleaning agent according to the invention comprises enzymes, preferably amylase, pectinase, carboanhydrase, tannase, lipase, mannanase, protease and/or cellulase, advantageously in quantities of from 0.0001-5 wt. % relative to the total agent. A suitable minimum quantity may also be 0.001 wt. % or 0.01 wt. %, relative to the total agent. Enzymes which may preferably be used are described further below in greater detail.

Furthermore, it is very particularly preferred for the washing or cleaning agent according to the invention to contain a builder system, preferably a zeolite-containing builder system, preferably comprising zeolite in quantities >5 wt. %, still more advantageously >10 wt. %, yet more advantageously >15 wt. %, in particular ≧20 wt. %, wt. % relating to the total agent. A sensible upper limit for zeolite may be for example 60 wt. %, 50 wt. % or 40 wt. %, relative to the total agent. This corresponds to a preferred embodiment of the invention.

For the purposes of the invention the term “builder system” also includes such “systems” which consist of only a single builder, such as for example zeolite. It is preferable, however, for at least 2 substances with a builder action to be used, for example zeolite in combination with soda, or the like.

It is likewise particularly preferable for the washing or cleaning agent according to the invention to contain a soluble builder system, preferably comprising soda, silicate, citrate and/or polycarboxylates, advantageously in quantities of from 0.1-50 wt. % relative to the total agent. This corresponds to a preferred embodiment of the invention. If such a soluble builder system is included, it is extremely preferable for only small quantities of insoluble builders, such as in particular zeolite, for example <5 wt. % to 0.1 wt. %, to be included, in particular in such a case for no insoluble builder to be included.

It is likewise possible for the washing or cleaning agent according to the invention to contain a builder system containing phosphates, in which phosphate is preferably included in quantities of from 1-40 wt. %, in particular 5-30 wt. %, relative to the total agent. According to another preferred embodiment, however, the washing or cleaning agent according to the invention contains no phosphates. Builders or builder systems which may preferably be used are described further below in greater detail.

Preferred washing or cleaning agents according to the invention have a pH>7.5, measured in a 5% solution of the agent in water at 20° C. However, washing or cleaning agents with a pH≦7.5, measured in a 5% solution of the agent in water at 20° C., are also possible, in particular liquid washing or cleaning agents.

The washing or cleaning agent according to the invention may, according to a further preferred embodiment of the invention, be in liquid or solid form, but preferably solid form, in particular it may be powdered or granular, advantageously it may be in the form of a molding, in particular a tablet.

The present invention also provides a textile washing method using a washing or cleaning agent according to the invention, in which the washing temperature amounts to <60° C., preferably ≦40° C. It is particularly advantageous that even at low temperatures, for example ≦40° C. or ≦30° C., very good cleaning performances may be achieved, in particular with regard to soiling attributable to engine oil. It is of course also possible to select higher washing temperatures than 60° C., for example 90° C. Then too, very good cleaning performances are achieved. However, such high temperatures are not necessary at all according to the invention in order to achieve excellent cleaning performances, such that energy may be saved. The textile washing method may be performed either manually or automatically, i.e. in an automatic washing machine.

The present invention accordingly also provides the use of a washing or cleaning agent according to the invention, as previously described, in the case of manual or automatic laundering of textiles.

If this use according to the invention is directed at the laundering of cotton-based textiles, synthetic fiber-based textiles, preferably polyester, and/or blend fabric-based textiles, in particular polyester/cotton blend fabric, a preferred embodiment of the invention is provided. The wholly surprising effect of the improved cleaning performance may be found in particular in the case of cotton-based textiles, synthetic fiber-based textiles, preferably polyester, and/or blend fabric-based textiles, in particular polyester/cotton blend fabric.

The invention further provides use of an amidine compound according to Formula (1)—

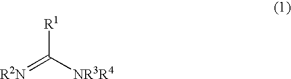

and/or of an amidinium bicarbonate according to Formula (2)—

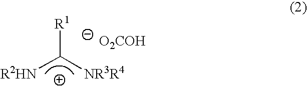

wherein R¹, R², R³, R⁴ mutually independently denote C₁-C₂₅ hydrocarbon residues or H, in washing or cleaning agents for improving dirt detachment in particular in the case of pigment dirt, preferably in the case of water-insoluble, polar pigment dirt, in particular garden soil.

Preferably, at least one of the residues R¹, R², R³ or R⁴, in particular the residue R², is a C₆-C₁₈ hydrocarbon residue, preferably C₈-C₁₈ hydrocarbon residue, the other residues R¹, R², R³ or R⁴, which are not C₆-C₁₈ hydrocarbon residues, being mutually independently preferably C₁-C₄ hydrocarbon residues or H.

In addition, the invention further provides the use of the just-mentioned compounds (1) and/or (2) in washing or cleaning agents for reducing soil adherence to the textile fibers to be cleaned, in particular in the case of pigment dirt, preferably in the case of water-insoluble, polar pigment dirt, in particular garden soil.

The invention likewise further provides use of an amidine compound according to Formula (1)—

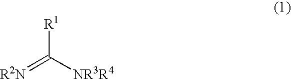

and/or of an amidinium bicarbonate according to Formula (2)—

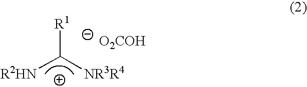

wherein R¹, R², R³, R⁴ mutually independently denote C₁-C₂₅ hydrocarbon residues or H, in washing or cleaning agents for improving dirt detachment preferably in the case of grease and oil soiling, in particular engine oil, makeup and sebaceous matter but above all engine oil.

Preferably, at least one of the residues R¹, R², R³ or R⁴, in particular the residue R², is a C₆-C₁₈ hydrocarbon residue, preferably C₈-C₁₈ hydrocarbon residue, the other residues R¹, R², R³ or R⁴, which are not C₆-C₁₈ hydrocarbon residues, being mutually independently preferably C₁-C₄ hydrocarbon residues or H.

In addition, the invention further provides use of the just-mentioned compounds (1) and/or (2) in washing or cleaning agents for reducing soil adherence to the textile fibers to be cleaned, preferably in the case of grease and oil soiling, in particular engine oil, makeup and sebaceous matter but above all engine oil.

The invention further provides a washing or cleaning method using a washing or cleaning agent containing an amidine compound according to Formula (1)—

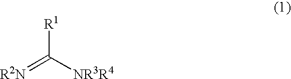

wherein R¹, R², R³, R⁴ are mutually and independently C₁-C₂₅ hydrocarbon residues or H, wherein carbon dioxide is absorbed from the ambient air into the washing liquor and is bound therein, forming the corresponding amidinium bicarbonate according to Formula (2)—

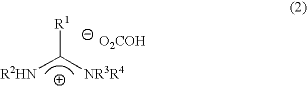

wherein R¹, R², R³, R⁴ correspond to those of the starting amidine compound (I). Preferably, at least one of the residues R¹, R², R³ or R⁴, particularly R², is a C₆-C₁₈ hydrocarbon residue, preferably C₈-C₁₈ hydrocarbon residue, the other residues R¹, R², R³ or R⁴, which are not C₆-C₁₈ hydrocarbon residues, being mutually independently preferably C₁-C₄ hydrocarbon residues or H.

The invention likewise further provides use of an amidine compound according to Formula (1)—

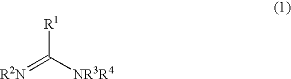

wherein R¹, R², R³, R⁴ are mutually and independently C₁-C₂₅ hydrocarbon residues or H, in a washing liquor in order to absorb carbon dioxide from the ambient air into the washing liquor and bind it therein, forming corresponding amidinium bicarbonate according to Formula (2)—

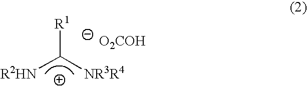

wherein R¹, R², R³, R⁴ correspond to those of the starting amidine compound (I).

Preferably, at least one of the residues R¹, R², R³ or R⁴, in particular the residue R², is a C₆-C₁₈ hydrocarbon residue, preferably C₈-C₁₈ hydrocarbon residue, the other residues R¹, R², R³ or R⁴, which are not C₆-C₁₈ hydrocarbon residues, being mutually independently preferably C₁-C₄ hydrocarbon residues or H.

The washing or cleaning agents usable according to the invention, which may for example assume the form in particular of powdered solids, post-compacted particles, homogeneous solutions or suspensions, may in principle additionally contain any ingredients which are known and conventional in such agents. The agents according to the invention may, as has already been disclosed, in particular contain builder substances, surface-active surfactants, furthermore also bleaching agents based on organic and/or inorganic peroxy compounds, bleaching activators, water-miscible organic solvents, enzymes, sequestering agents, electrolytes, pH regulators and further auxiliary materials, such as optical brighteners, graying inhibitors, foam regulators together with colorants and fragrances.

The washing or cleaning agents according to the invention, as already explained, may contain one surfactant or two or more surfactants, it being possible in particular to consider not only anionic surfactants, nonionic surfactants and mixtures thereof, but also cationic, zwitterionic and amphoteric surfactants.

Suitable nonionic surfactants are in particular alkylglycosides and ethoxylation and/or propoxylation products of alkylglycosides or linear or branched alcohols in each case having 12 to 18 C atoms in the alkyl moiety and 3 to 20, preferably 4 to 10, alkyl ether groups. Corresponding ethoxylation and/or propoxylation products of N-alkylamines, vicinal diols, fatty acid esters and fatty acid amides, which correspond with regard to the alkyl moiety to the stated long-chain alcohol derivatives, and of alkylphenols having 5 to 12 C atoms in the alkyl residue may furthermore be used.

Preferably used nonionic surfactants are alkoxylated, advantageously ethoxylated, in particular primary alcohols with preferably 8 to 18 C atoms and on average 1 to 12 mol ethylene oxide (EO) per mol of alcohol, in which the alcohol residue may be linear or preferably methyl-branched in position 2 or may contain linear and methyl-branched residues in the mixture, as they are usually present in oxo alcohol residues. In particular, however, alcohol ethoxylates with linear residues prepared from alcohols of natural origin with 12 to 18 C atoms, for example from coconut, palm, tallow fat or oleyl alcohol, and on average 2 to 8 EO per mol of alcohol are preferred. Preferred ethoxylated alcohols include, for example, C₁₂-C₁₄ alcohols with 3 EO or 4 EO, C₉-C₁₁ alcohols with 7 EO, C₁₃-C₁₅ alcohols with 3 EO, 5 EO, 7 EO or 8 EO, C₁₂-C₁₈ alcohols with 3 EO, 5 EO or 7 EO and mixtures of these, such as mixtures of C₁₂-C₁₄ alcohol with 3 EO and C₁₂-C₁₈ alcohol with 7 EO. The stated degrees of ethoxylation are statistical averages which, for a specific product, may be an integer or a fractional number. Preferred alcohol ethoxylates have a narrow homolog distribution (narrow range ethoxylates, NRE). In addition to these nonionic surfactants, fatty alcohols with more than 12 BO may also be used. Examples of these are (tallow) fatty alcohols with 14 EO, 16 EO, 20 EO, 25 EO, 30 EO or 40 EO. In particular in agents for use in machine washing methods, extremely low-foam compounds are conventionally used. These preferably include C₁₂-C₁₈ alkyl polyethylene glycol/polypropylene glycol ethers having up to about 8 mol of ethylene oxide and propylene oxide units per molecule. It is, however, also possible to use other nonionic surfactants which are known to be low-foaming, such as for example C₁₂-Cᵢₛ-alkyl polyethylene glycol/polybutylene glycol ethers with in each case up to 8 mol ethylene oxide and butylene oxide units per molecule and end group-terminated alkyl polyalkylene glycol mixed ethers. The alkoxylated alcohols containing hydroxyl groups as described in European patent application EP 0 300 305, or “hydroxy mixed ethers”, are also particularly preferred. Alkyl glycosides of the general formula RO(G)ₓ, in which R means a primary straight-chain or methyl-branched aliphatic residue, in particular methyl-branched in position 2, with 8 to 22, preferably 12 to 18 C atoms and G denotes a glycose unit with 5 or 6 C atoms, preferably glucose, may also be used as nonionic surfactants. The degree of oligomerization x, which indicates the distribution of monoglycosides and oligoglycosides, is any desired number (which, being an analytically determined variable, may also assume fractional values) between 1 and 10; x is preferably 1.2 to 1.4. Also suitable are polyhydroxy fatty acid amides according to Formula (3), in which R¹CO denotes an aliphatic acyl residue with 6 to 22 carbon atoms, R² denotes hydrogen, an alkyl or hydroxyalkyl residue with 1 to 4 carbon atoms, and [Z] denotes a linear or branched polyhydroxyalkyl residue with 3 to 10 carbon atoms and 3 to 10 hydroxyl groups—

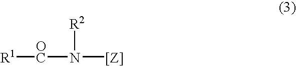

Polyhydroxy fatty acid amides are preferably derived from reducing sugars with 5 or 6 carbon atoms, in particular from glucose. The group of polyhydroxy fatty acid amides also includes compounds according to Formula (4)—

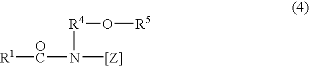

wherein R³ is a linear or branched alkyl or alkenyl residue with 7 to 12 carbon atoms, R⁴ is a linear, branched or cyclic alkylene residue or an arylene residue with 2 to 8 carbon atoms, R⁵ is a linear, branched or cyclic alkyl residue or an aryl residue or an oxyalkyl residue with 1 to 8 carbon atoms, C₁-C₄ alkyl or phenyl residues being preferred, and [Z] is a linear polyhydroxyalkyl residue, wherein the alkyl chain is substituted with at least two hydroxyl groups, or alkoxylated, preferably ethoxylated or propoxylated, derivatives of this residue. [Z] is also preferably obtained by reductive amination of a sugar such as glucose, fructose, maltose, lactose, galactose, mannose or xylose. N-alkoxy- or N-aryloxy-substituted compounds may then, for example, be converted into desired polyhydroxyfatty acid amides by reaction with fatty acid methyl esters in the presence of an alkoxide as catalyst. A further class of preferably usable nonionic surfactants, which may be used either as sole nonionic surfactant or in combination with other nonionic surfactants, in particular together with alkoxylated fatty alcohols and/or alkylglycosides, comprises alkoxylated, preferably ethoxylated or ethoxylated and propoxylated fatty acid alkyl esters, preferably with 1 to 4 carbon atoms in the alkyl chain, in particular fatty acid methyl esters. Nonionic surfactants of the amine oxide type, for example N-coconut alkyl-N,N-dimethylamine oxide and N-tallow alcohol-N,N-dihydroxyethylamine oxide, and of the fatty acid alkanolamide type may also be suitable. The quantity of these nonionic surfactants preferably amounts to no more than that of the ethoxylated fatty alcohols, in particular no more than half the quantity thereof. “Gemini” surfactants may also be considered as further surfactants. These are generally taken to mean such compounds as have two hydrophilic groups per molecule. These groups are generally separated from one another by a “spacer”. This spacer is generally a carbon chain which should be long enough for the hydrophilic groups to be sufficiently far apart that they can act mutually independently. Such surfactants are in general distinguished by an unusually low critical micelle concentration and the ability to bring about a great reduction in the surface tension of water. In exceptional cases, gemini surfactants include not only such “dimeric” surfactants, but also corresponding “trimeric” surfactants. Suitable gemini surfactants are for example sulfated hydroxy mixed ethers or dimer alcohol bis- and trimer alcohol tris-sulfates and -ether sulfates. End group-terminated dimeric and trimeric mixed ethers are distinguished in particular by their di- and multifunctionality. The stated end group-terminated surfactants accordingly exhibit good wetting characteristics and are low-foaming, such that they are in particular suitable for use in machine washing or cleaning methods. Gemini polyhydroxyfatty acid amides or poly-polyhydroxyfatty acid amides may however also be used. The sulfuric acid monoesters of straight-chain or branched C₇-C₂₁ alcohols ethoxylated with 1 to 6 mol of ethylene oxide are also suitable, such as 2-methyl-branched C₉-C₁₁ alcohols with on average 3.5 mol of ethylene oxide (EO) or C₁₂-C₁₈ fatty alcohols with 1 to 4 EO. Preferred anionic surfactants also include the salts of alkylsulfosuccinic acid, which are also known as sulfosuccinates or sulfosuccinic acid esters, and are the monoesters and/or diesters of sulfosuccinic acid with alcohols, preferably fatty alcohols and in particular ethoxylated fatty alcohols. Preferred sulfosuccinates contain C₈ to C₁₈ fatty alcohol residues or mixtures thereof. Particularly preferred sulfosuccinates contain a fatty alcohol residue which is derived from ethoxylated fatty alcohols, which are in themselves nonionic surfactants. Sulfosuccinates whose fatty alcohol residues are derived from ethoxylated fatty alcohols with a narrow homologue distribution are here particularly preferred. It is likewise also possible to use alk(en)ylsuccinic acid with preferably 8 to 18 carbon atoms in the alk(en)yl chain or the salts thereof. Further anionic surfactants which may be considered are fatty acid derivatives of amino acids, for example of N-methyltaurine (taurides) and/or of N-methylglycine (sarcosides). Sarcosides or sarcosinates are particularly preferred here and most especially sarcosinates of higher and optionally mono- or polyunsaturated fatty acids such as oleyl sarcosinate. Further anionic surfactants which may in particular be considered are soaps. Saturated fatty acid soaps are in particular suitable, such as the salts of lauric acid, myristic acid, palmitic acid, stearic acid, hydrogenated erucic acid and behenic acid and in particular soap mixtures derived from natural fatty acids, for example coconut, palm kernel or tallow fatty acids. Known alkenylsuccinic acid salts may also be used together with these soaps or as substitutes for soaps.

The anionic surfactants, including the soaps, may be present in the form of the sodium, potassium or ammonium salts thereof and as soluble salts of organic bases, such as mono-, di- or triethanolamine. The anionic surfactants are preferably present in the form of the sodium or potassium salts thereof, in particular in the form of the sodium salts.

Suitable surfactant quantities for use have already been stated above. They are present in washing or cleaning agents according to the invention for example in proportions of preferably 5 wt. % to 50 wt. %, in particular of 8 wt. % to 30 wt. %, wt. % relating to the total agent.

A washing or cleaning agent according to the invention may preferably contain at least one water-soluble and/or water-insoluble, organic and/or inorganic builder. Water-soluble organic builder substances include polycarboxylic acids, in particular citric acid and saccharic acids, monomeric and polymeric aminopolycarboxylic acids, in particular methylglycinediacetic acid, nitrilotriacetic acid and ethylenediaminetetraacetic acid together with polyaspartic acid, polyphosphonic acids, in particular aminotris(methylenephosphonic acid), ethylenediaminetetrakis(methylenephosphonic acid) and 1-hydroxyethane-1,1-diphosphonic acid, polymeric hydroxyl compounds such as dextrin and polymeric (poly-)carboxylic acids, in particular the polycarboxylates accessible by oxidizing polysaccharides or dextrins, polymeric acrylic acids, methacrylic acids, maleic acids and copolymers thereof, which may also contain small proportions of polymerizable substances without carboxylic acid functionality incorporated by polymerization. The relative molecular mass of the homopolymers of unsaturated carboxylic acids is in general between 3000 and 200000, that of the copolymers between 2000 and 200000, preferably 30000 to 120000, in each case relative to free acid. One particularly preferred acrylic acid/maleic acid copolymer has a relative molecular mass of 30000 to 100000. Conventional commercial products are for example Sokalan® CP 5, CP 10 and PA 30 from BASF. Suitable, albeit less preferred, compounds of this class are copolymers of acrylic acid or methacrylic acid with vinyl ethers, such as vinyl methyl ethers, vinyl esters, ethylene, propylene and styrene, the acid fraction of which amounts to at least 50 wt. %. Terpolymers containing as monomers two unsaturated acids and/or the salts thereof and, as third monomer, vinyl alcohol and/or an esterified vinyl alcohol or a carbohydrate may also be used as water-soluble organic builder substances. The first acidic monomer or the salt thereof is derived from a monoethylenically unsaturated C₃-C₈ carboxylic acid and preferably from a C₃-C₄ monocarboxylic acid, in particular from (meth)acrylic acid. The second acidic monomer or the salt thereof may be a derivative of a C₄-C₈ dicarboxylic acid, maleic acid being particularly preferred, and/or a derivative of an allylsulfonic acid which is substituted in position 2 with an alkyl or aryl residue. Such polymers generally have a relative molecular mass of between 1000 and 200000. Further preferred copolymers are those which preferably comprise acrolein and acrylic acid/acrylic acid salt or vinyl acetate as monomers. The organic builder substances may be used, in particular for producing liquid agents, in the form of aqueous solutions, preferably in the form of 30 to 50 wt. % aqueous solutions. All the stated acids are generally used in the form of the water-soluble salts, in particular the alkali metal salts, thereof.

Such organic builder substances may be used according to a preferred embodiment of the invention and may, if desired, be present in quantities of up to 40 wt. %, in particular of up to 25 wt. % and preferably of 1 wt. % to 8 wt. %. Quantities close to the stated upper limit are preferably used in pasty or liquid, in particular water-containing, agents according to the invention.

Water-soluble inorganic builder materials which may in particular be considered are alkali metal silicates, alkali metal carbonates and alkali metal phosphates, which may be present in the form of the alkaline, neutral or acidic sodium or potassium salts thereof. Examples of these are trisodium phosphate, tetrasodium diphosphate, disodium dihydrogendiphosphate, pentasodium triphosphate, “sodium hexametaphosphate”, oligomeric trisodium phosphate with degrees of oligomerization of 5 to 1000, in particular 5 to 50, and the corresponding potassium salts or mixtures of sodium and potassium salts. Water-insoluble, water-dispersible inorganic builder materials which are used are in particular crystalline or amorphous alkali metal aluminosilicates, in quantities of up to 50 wt. %, preferably of no more than 40 wt. % and, in liquid agents, in particular from 1 wt. % to 5 wt. %. Preferred such materials are crystalline sodium aluminosilicates of washing agent grade, in particular zeolite A, P and optionally X, alone or in mixtures, for example in the form of a co-crystallization product of zeolites A and X (Vegobond® AX, a commercial product from Condea Augusta S.p.A). Quantities close to the stated upper limit are preferably used in solid, particulate agents. Suitable aluminosilicates in particular comprise no particles with a grain size of above 30 μm and preferably consist to an extent of at least 80 wt. % of particles with a size below 10 μm. Their calcium binding capacity, which may be determined as stated in German patent DE 24 12 837, is generally in the range of from 100 to 200 mg of CaO per gram.

Suitable substitutes or partial substitutes for the stated aluminosilicate are crystalline alkali metal silicates, which may be present alone or mixed with amorphous silicates. The alkali metal silicates usable as builders in the agents according to the invention preferably have a molar ratio of alkali metal oxide to SiO₂ of below 0.95, in particular of 1:1.1 to 1:12 and may be in amorphous or crystalline form. Preferred alkali metal silicates are sodium silicates, in particular amorphous sodium silicates, with a molar ratio Na₂O:SiO₂ of 1:2 to 1:2.8. Preferably used crystalline silicates, which may be present alone or mixed with amorphous silicates, are crystalline phyllosilicates of the general formula Na₂SiₓO₂ₓ₊₁.yH₂O, in which x, the “modulus”, is a number from 1.9 to 22, in particular 1.9 to 4 and y is a number from 0 to 33 and preferred values for x are 2, 3 or 4. Preferred crystalline phyllosilicates are those in which x in the stated general formula assumes the values 2 or 3. In particular, both β- and δ-sodium disilicates (Na₂Si₂O₅.yH₂O) are preferred. Virtually anhydrous crystalline alkali metal silicates, produced from amorphous alkali metal silicates, of the above-stated general formula in which x means a number from 1.9 to 2.1 may also be used in agents according to the invention. A crystalline sodium phyllosilicate with a modulus of 2 to 3, as may be produced from sand and soda, is used in a further preferred embodiment of agents according to the invention. Crystalline sodium silicates with a modulus in the range from 1.9 to 3.5 are used in a further preferred embodiment of agents according to the invention. Crystalline layered silicates of the above-stated formula (1) are sold by Clariant GmbH under the trade name Na—SKS, for example Na—SKS-1 (Na₂Si₂₂O₄₅.xH₂O, kenyaite), Na—SKS-2 (Na₂Si₁₄O₂₉.xH₂O, magadiite), Na—SKS-3 (Na₂Si₈O₁₇.xH₂O) or Na—SKS-4 (Na₂Si₄O₉.xH₂O, makatite). Of these Na—SKS-5 (α-Na₂Si₂O₅), Na—SKS-7 (β-Na₂Si₂O₅, natrosilite), Na—SKS-9 (NaHSi₂O₅.3H₂O), Na—SKS-10 (NaHSi₂O₅.3H₂O, kanemite), Na—SKS-11 (t-Na₂Si₂O₅) and Na—SKS-13 (NaHSi₂O₅) are particularly suitable, in particular however Na—SKS-6 (5-Na₂Si₂O₅). In a preferred development of agents according to the invention, a granular compound is used which is prepared from crystalline phyllosilicate and citrate, from crystalline phyllosilicate and above-stated (co)polymeric polycarboxylic acid or from alkali metal silicate and alkali metal carbonate, as is commercially available for example under the name Nabion® 15.

Builder substances may be present in the washing or cleaning agents according to the invention preferably in quantities of up 75 wt. %, in particular of 5 wt. % to 50 wt. %, wt. % relating to the total agent.

Peroxy compounds suitable for use in washing or cleaning agents according to the invention which may in particular be considered are organic peracids or peracid salts of organic acids, such as phthalimidopercaproic acid, perbenzoic acid or salts of diperdodecanedioic acid, hydrogen peroxide and inorganic salts which release hydrogen peroxide under washing conditions, which latter include perborate, percarbonate, persilicate and/or persulfate such as caroate. Where solid peroxy compounds are to be used, they may be used in the form of powders or granules, which may also in principle be encapsulated in known manner. If an agent according to the invention contains peroxy compounds, these are preferably present in quantities of up to 50 wt. %, in particular of 5 wt. % to 30 wt. %. It may be appropriate to add small quantities of known bleaching agent stabilizers, such as for example phosphonates, borates or metaborates and metasilicates and magnesium salts such as magnesium sulfate.

Bleaching activators which may be used are compounds which, under perhydrolysis conditions, yield aliphatic peroxycarboxylic acids with preferably 1 to 10 C atoms, in particular 2 to 4 C atoms, and/or optionally substituted perbenzoic acid. Suitable substances are those which bear O- and/or N-acyl groups having the stated number of C atoms and/or optionally substituted benzoyl groups. Preferred compounds are repeatedly acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate, 2,5-diacetoxy-2,5-dihydrofuran and enol ester and acetylated sorbitol and mannitol or mixtures thereof (SORMAN), acylated sugar derivatives, in particular pentaacetyl glucose (PAG), pentaacetyl fructose, tetraacetyl xylose and octaacetyl lactose and acetylated, optionally N-alkylated glucamine and gluconolactone, and/or N-acylated lactams, for example N-benzoylcaprolactam. The hydrophilically substituted acyl acetals and acyl lactams are likewise preferably used. Combinations of conventional bleaching activators may also be used. Such bleaching activators may be present, in particular in the presence of the above-stated hydrogen peroxide-releasing bleaching agents, in a conventional quantity range, preferably in quantities of 0.5 wt. % to 10 wt. %, in particular 1 wt. % to 8 wt. %, relative to the total agent, but are preferably entirely absent when percarboxylic acid is used as the sole bleaching agent.

In addition to or instead of conventional bleaching activators, sulfone imines and/or bleach-boosting transition metal salts or transition metal complexes may also be present as “bleach catalysts”.

Enzymes which may be used in the washing or cleaning agents according to the invention come above all from the class of amylases, proteases, lipases, cutinases, pullulanases, hemicellulases, cellulases, oxidases, laccases, pectinases, carboanhydrases, mannanases, tannases and peroxidases and mixtures thereof. Particularly suitable enzymatic active ingredients are those obtained from fungi or bacteria, such as Bacillus subtilis, Bacillus licheniformis, Bacillus lentus, Streptomyces griseus, Humicola lanuginosa, Humicola insolens, Pseudomonas pseudoalcaligenes, Pseudomonas cepacia or Coprinus cinereus. The enzymes may be adsorbed onto carrier substances and/or be embedded in encapsulating substances in order to protect them from premature inactivation They are present in the washing or cleaning agents according to the invention preferably in quantities of up to 5 wt. %, in particular of 0.2 wt. % to 4 wt. %. If the agent according to the invention contains protease, it preferably exhibits a proteolytic activity in the range from approx. 100 PU/g to approx. 10000 PU/g, in particular 300 PU/g to 8000 PU/g. If two or more enzymes are to be used in the agent according to the invention, this may be achieved by incorporating the two or more separate enzymes or enzymes which are separately formulated in known manner or by two or more enzymes jointly formulated in a granular product.

Organic solvents other than water which may be used in the washing or cleaning agents according to the invention, in particular if these are in liquid or pasty form, include alcohols with 1 to 4 C atoms, in particular methanol, ethanol, isopropanol and tert.-butanol, diols with 2 to 4 C atoms, in particular ethylene glycol and propylene glycol, and mixtures thereof and the ethers derivable from the stated classes of compounds. Such water-miscible solvents are preferably present in the agents according to the invention in quantities of no more than 30 wt. %, in particular of 6 wt. % to 20 wt. %.

In order to establish a desired pH value which is not automatically obtained by mixing the remaining components, the agents according to the invention may contain acids which are compatible with the system and are environmentally compatible, in particular citric acid, acetic acid, tartaric acid, malic acid, lactic acid, glycolic acid, succinic acid, glutaric acid and/or adipic acid, as well as mineral acids, in particular sulfuric acid, or bases, in particular ammonium or alkali metal hydroxides. Such pH regulators are present in the agents according to the invention in quantities of preferably no more than 20 wt. %, in particular of 1.2 wt. % to 17 wt. %.

Graying inhibitors have the task of keeping dirt which has been dissolved away from the textile fiber suspended in the liquor. Water-soluble colloids of a mainly organic nature are suitable for this purpose, for example starch, size, gelatin, salts of ether carboxylic acids or ether sulfonic acids of starch or cellulose or salts of acidic sulfuric acid esters of cellulose or starch. Water-soluble polyamides containing acidic groups are also suitable for this purpose. Derivatives of starch other than those stated above, for example aldehyde starches, may further be used. Cellulose ethers, such as carboxymethylcellulose (Na salt), methylcellulose, hydroxyalkylcellulose and mixed ethers, such as methylhydroxyethylcellulose, methylhydroxypropylcellulose, methylcarboxymethylcellulose and mixtures thereof, are preferably used, for example in quantities of 0.1 to 5 wt. % relative to the agents.

Washing or cleaning agents according to the invention may for example contain derivatives of diaminostilbene disulfonic acid or the alkali metal salts thereof as optical brighteners, although they preferably contain no optical brighteners for use as a color washing agent. Suitable compounds are, for example, salts of 4,4′-bis(2-anilino-4-morpholino-1,3,5-triazinyl-6-amino)stilbene 2,2′-disulfonic acid or compounds of similar structure which, instead of the morpholino group, bear a diethanolamino group, a methylamino group, an anilino group or a 2-methoxyethylamino group. Brighteners of the substituted diphenylstyryl type may furthermore be present, for example the alkali metal salts of 4,4′-bis(2-sulfostyryl)-diphenyl, 4,4′-bis(4-chloro-3-sulfostyryl)-diphenol, or 4-(4-chlorostyryl)-4′-(2-sulfostyryl)-diphenyl. Mixtures of the above-stated optical brighteners may also be used.

Especially for use in machine washing methods, it may be advantageous to add conventional foam inhibitors to the agents. Suitable foam inhibitors are, for example, soaps of natural or synthetic origin, which comprise an elevated proportion of C₁₈-C₂₄ fatty acids. Suitable non-surfactant foam inhibitors are, for example, organopolysiloxanes and mixtures thereof with microfine, optionally silanized silica as well as paraffins, waxes, microcrystalline waxes and mixtures thereof with silanized silica or bis-fatty acid alkylenediamides. Mixtures of different foam inhibitors are also advantageously used, for example mixtures of silicones, paraffins or waxes. The foam inhibitors, in particular foam inhibitors containing silicone and/or paraffin, are preferably bound to a granular carrier substance which is soluble or dispersible in water. Mixtures of paraffins and bistearylethylenediamide are particularly preferred here.

The production of solid washing or cleaning agents according to the invention presents no difficulties and may proceed in known manner, for example by spray drying or granulation, with enzymes and any further thermally sensitive ingredients such as for example bleaching agents optionally subsequently being separately added. Incorporation of the amidine compound to be used according to the invention and/or of the amidinium bicarbonate according to the invention may proceed for example together with other ingredients, such as for example with the other surfactants, if other surfactants are used. Agents according to the invention with an elevated bulk density, in particular in the range from 650 g/l to 950 g/l, may preferably be produced by a method comprising an extrusion step. Production of the amidine compounds and amidinium bicarbonates usable according to the invention is described in detail in international patent application WO 2007/056859 A1. Reference is here made to the content of international patent application WO 2007/056859 A1 in its entirety.

Agents according to the invention may preferably be produced in the form of tablets, which may be monophasic or multiphasic, single-colored or multicolored and in particular consist of one layer or of two or more, in particular two, layers, by mixing together all the components, optionally for each layer, in a mixer and compression molding the mixture by means of conventional tablet presses, for example eccentric presses or rotary presses, with pressing forces in the range from approx. 50 to 100 kN, preferably 60 to 70 kN. In particular in the case of multilayer tablets, it may be advantageous for at least one layer to be preliminarily compression molded. This is preferably carried out at pressing forces of between 5 and 20 kN, in particular of 10 to 15 kN. In this manner, break-resistant tablets are straightforwardly obtained which nevertheless dissolve sufficiently rapidly under conditions of use and exhibit breaking and flexural strength values usually of 100 to 200 N, but preferably of above 150 N. A tablet produced in this manner is preferably of a weight of 10 g to 50 g, in particular of 15 g to 40 g. The tablets may be of any desired three-dimensional shape and may be round, oval or polygonal, intermediate shapes also being possible. Corners and edges are advantageously rounded. Round tablets preferably have a diameter of 30 mm to 40 mm. In particular the size of polygonal or cuboidal tablets, which are predominantly introduced by means of the dispenser for example of a dishwashing machine, is dependent on the geometry and volume of this dispenser. Preferred embodiments have, for example, a base area of (20 to 30 mm)ₓ(34 to 40 mm), in particular, 26×36 mm or 24×38 mm.

Liquid or pasty agents according to the invention in the form of solutions containing conventional solvents are generally produced by simply mixing the ingredients, which may be introduced into an automatic mixer as an undissolved material or as a solution.

### EXAMPLES

### Examples According to the Invention

Washing agent E1 according to the invention was a conventional, powdered color washing agent (corresponding to 100 parts by weight), to which 2 parts by weight of the amidine compound ( [symbol] ) were additionally added.

The amidine compound ( [symbol] ) complied with the following formula—

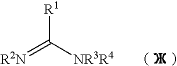

R¹, R³ and R⁴ were each methyl residues. R² was a saturated, straight-chain C₁₋₂ hydrocarbon residue.

### Comparative Example 1

Comparative washing agent V1 corresponded to washing agent E1. However, instead of the amidine compound ( [symbol] ), it additionally contained 2 parts by weight of LAS.

### Washing Tests—

The above-stated agents E1 and V1 were tested under the same conditions using cotton textiles soiled with engine oil.

Washing conditions were as follow—

Colored wash program, 40° C./Miele W 985

Number of washing/drying cycles: 2

Water hardness: 16 German hardness degrees

Rate of addition: 75 g

### Results—

Cleaning performance of washing agent E1 according to the invention was markedly better than that of comparative washing agent V1.

We claim:

1. Washing or cleaning agent comprising: an amidine compound according to Formula (1)— 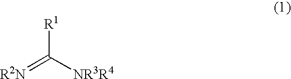
and/or an amidinium bicarbonate according to Formula (2)— 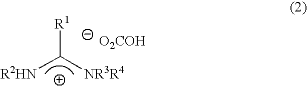
wherein R¹, R², R³, R⁴ are mutually and independently C₁-C₂₅ hydrocarbon residues or H.

2. Agent according to claim 1, wherein the amidine compound according to Formula (1) and/or the amidinium bicarbonate according to Formula (2) is present in the agent in an amount of 0.1 wt. % to 25 wt. %, based on total weight of the agent.

3. Agent according to claim 1 further comprising anionic, nonionic and/or cationic surfactants present in an amount of from 0.1 wt. % to 50 wt. %, based on total weight of the agent.

4. Agent according to claim 3, wherein the surfactant is at least an alkylbenzene sulfonate present in an amount of from 0.1 wt. % to 25 wt. %, based on total weight of the agent.

5. Agent according to claim 3, wherein the surfactant is at least an alkyl sulfate present in an amount of from 0.1 wt. % to 25 wt. %, based on total weight of the agent.

6. Agent according to claim 3, wherein the surfactant is at least a nonionic surfactant present in an amount of from 0.01 wt. % to 20 wt. %, based on total weight of the agent.

7. Agent according to claim 1 further comprising enzymes present in an amount of from 0.0001 wt. % to 5 wt. %, based on total weight of the agent.

8. Agent according to claim 1, wherein the agent is a solid agent in the form of a tablet.

9. Method of washing textiles comprising adding to a wash an agent according to claim 1, and washing the textiles at a temperature of 60° C. or less.

10. Method according to claim 9, wherein the textiles laundered are natural fiber textiles, synthetic fiber textiles, blends of natural and synthetic fiber textiles, or mixtures thereof.

11. Method according to claim 9, further comprising absorbing carbon dioxide from ambient air into the wash, binding the carbon dioxide therein, thereby forming the corresponding amidinium bicarbonate according to Formula (2).
